(19) United States
(12) Patent Application Publication
(10) Pub. No.: US 2011/0226651 A1
(43) Pub. Date: Sep. 22, 2011

(54) Medicine Container Cover

(76) Inventor: Erica Patino, Houston, TX (US)

(21) Appl. No.: 13/046,459

(22) Filed: Mar. 11, 2011

Related U.S. Application Data
(60) Provisional application No. 61/312,937, filed on Mar. 11, 2010.

(51) Int. Cl. A61B 17/00; B65B 5/00

(52) U.S. Cl. 206/438; 53/467

(57) ABSTRACT

A medicine cover for covering a medicine container and a method for using the same is provided. The medicine cover may have a cylindrical sleeve, or a flat medicine cover, for placing over the medicine container and concealing the identity of the medicine container. The medicine cover may have a bottom having an attachment device for securing the bottom to the cylindrical sleeve.

### CROSS-REFERENCE TO RELATED APPLICATIONS

This application claims the benefit of U.S. Provisional Application No. 61/312,937 filed Mar. 11, 2010 which is herein incorporated by reference in its entirety.

### BACKGROUND

Embodiments of the inventive subject matter generally relate to the field of bottle covers, more particularly to a cover for placing over a prescription pill bottle to cover the bottle. The bottle may have a notification device to alert the owner of the time to take the pill.

### SUMMARY

Embodiments described herein are a medicine cover and method for using the same. The medicine cover may be for covering a medicine container. The medicine cover may have a cylindrical sleeve for placing over the medicine container and concealing the identity of the medicine container and a bottom having an attachment device for securing the bottom to the cylindrical sleeve. The medicine container may hold a medicine for a patient to take that is stored in the medicine container.

Embodiments described herein further comprise a notification device configured to provide a medicine alert to the patient. The medicine alert may alert when the patient should take the medicine. The notification device may be a clock and the medicine alert is an audio alarm heard by the patient. The notification device may be in communication with a communication network. The notification device may send the medicine alert to the patient's phone. The notification device may send the medicine alert to pharmacy.

Embodiments described herein further comprise a medicine data unit configured to store data regarding the medicine alert. The medicine data unit may determine when the medicine is running low and alert a pharmacy to refill the medicine.

Embodiments described herein further comprise the bottom being a removable bottom having a fastener. The fastener may be a threaded connection, a magnetic connection, and the like.

Embodiments described herein further comprise the cylindrical sleeve of the medicine cover being made of neoprene.

Embodiments described herein may be an apparatus for concealing a medicine. The apparatus may have a medicine cover, a medicine container for storing the medicine, and an indicator for sending one or more medicine alerts about the medicine. The medicine cover may further comprise a flat medicine cover having a lid foldable over a medicine holder portion of the flat medicine cover. The medicine may be on a sheet, or in pill form. The medicine may be a birth control dispenser. The indicator may be in communication with a medicine system. The medicine system may have a communication network and a patient's computer. The patient's computer may be a personal digital assistant. The medicine system may further comprise a doctor's office, and/or a pharmacy. The medicine cover may further have a sensor configured to indicate when the medicine is being taken.

Embodiments described herein may be a method for taking medicine. The method may comprise placing a medicine in a medicine container and covering a portion of the medicine container with a medicine cover. The method may further comprise hiding the identity of the medicine with the medicine cover and taking the medicine at a required time. The method may further comprise providing a notification device on the medicine cover and notifying a patient of the required time with the notification device. The method may further comprise sending a text message to the patient. The method may further comprise sending an email to the patient. The method may further comprise setting the required time, wherein setting the required time further comprises the patient setting a clock on the medicine cover. The method may further comprise setting the required time further comprises the pharmacy sending data to an indicator on the medicine cover. The method wherein setting the required time further comprises a doctor sending data to an indicator on the medicine cover. The method may further comprise sensing when a patient takes the medicine. The method may further comprise ordering the medicine cover on a website. The method may further comprise indicating the type of medicine to be covered and the required time to take the medicine.

### BRIEF DESCRIPTION OF THE DRAWINGS

The present embodiments may be better understood, and numerous objects, features, and advantages made apparent to those skilled in the art by referencing the accompanying drawings. These drawings are used to illustrate only typical embodiments of this invention, and are not to be considered limiting of its scope, for the invention may admit to other equally effective embodiments. The figures are not necessarily to scale and certain features and certain views of the figures may be shown exaggerated in scale or in schematic in the interest of clarity and conciseness.

FIG. 1 depicts a schematic of a medicine system according to one embodiment.

FIG. 2 depicts a perspective view of a medicine cover covering a medicine container, according to an embodiment.

FIG. 3 depicts a back view of the medicine cover without the medicine container.

FIG. 4 depicts a front view of the medicine cover without the medicine container.

FIG. 5 depicts a perspective exploded view of the medicine cover having a removable bottom with a threaded connection.

FIG. 6 depicts a perspective exploded view of the medicine cover having the removable bottom with a magnetic connection.

FIG. 7 depicts the medicine cover as a flat medicine cover according to an embodiment.

FIG. 8 depicts an end view of the flat medicine cover according to an embodiment.

FIG. 9 depicts a front view of the flat medicine cover according to an embodiment.

FIG. 10 depicts a perspective view of the flat medicine cover in an open position according to an embodiment.

FIG. 11 depicts a block diagram depicting a method of using the medicine system as shown in FIG. 1.

### DETAILED DESCRIPTION OF EMBODIMENT(S)

The description that follows includes exemplary apparatus, methods, techniques, and instruction sequences that embody techniques of the present inventive subject matter. However, it is understood that the described embodiments may be practiced without these specific details.

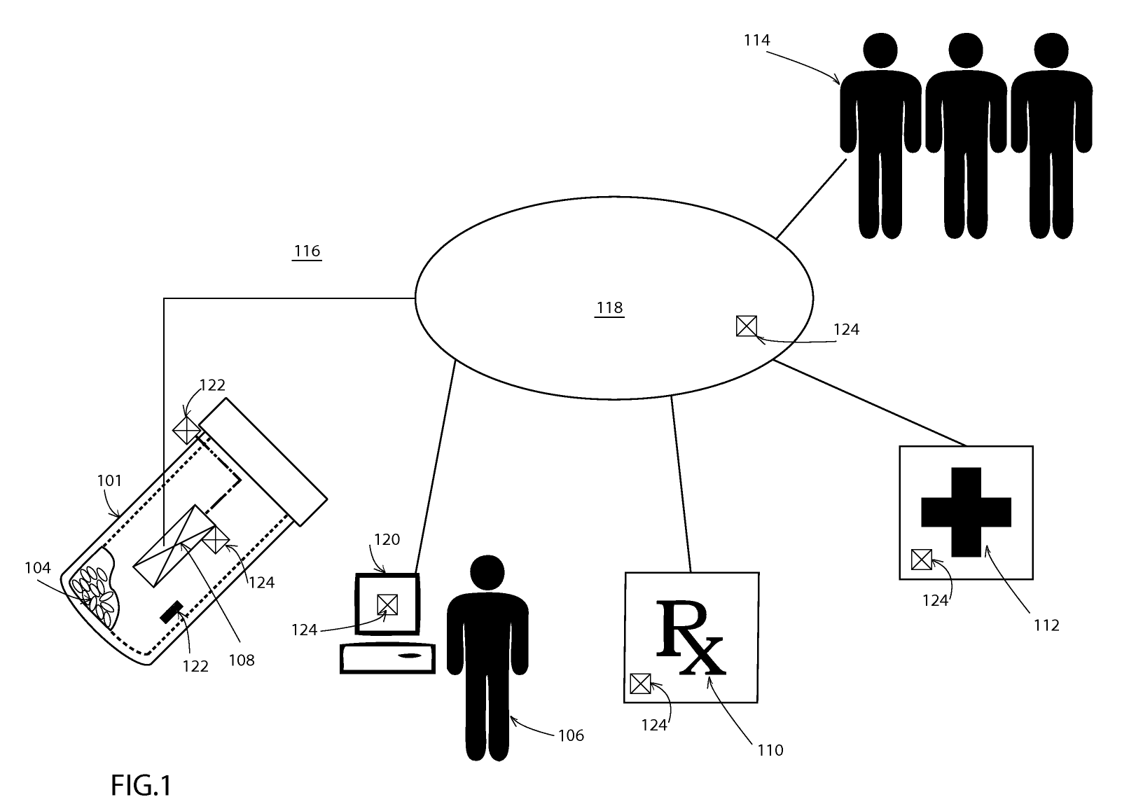

FIG. 1 depicts a schematic of a medicine system 100 according to one embodiment. The medicine system 100 may have a medicine cover 101. The medicine cover 101 may be configured to cover a medicine container 102. The medicine container 102 may be configured to hold medicine 104. The medicine cover 101 may be configured to partially cover the medicine container 102 (as shown) or cover the entire medicine container 102. The medicine cover 101 may conceal the medicine container 102 thereby concealing the identity of the medicine 104, or the entire medicine container 102. This may allow a patient 106 discreetly carry the medicine container 102 while concealing the medicine 104 type and/or quantity. The medicine cover 101 may have an indicator 108, or notification device. The indicator 108 may alert the patient 106 when it is time to take the medicine 104. Further, the indicator 108 may in communication with one or more pharmacies 110, one or more doctors 112, and one or more third parties 114 in addition to alerting the patient 106.

The indicator 108 may be in communication with a system 116 for alerting the patient 106, the pharmacies 110, the doctors 112 and/or the third parties 114 about the time to take the medicine 104, and/or the usage of the medicine 104. In one embodiment, the indicator 108 may be in wireless communication with a communication network 118. The communication network 118 may allow for communication between the indicator 108, the pharmacy 110, the doctor 112, a patient computer 120 and/or the one or more third parties 114. The communication network 118 may communicate with any of the devices and systems, using any combination of communication devices or methods such as, wired, telemetry, wireless, fiber optics, acoustic, infrared, a local area network (LAN), a personal area network (PAN), and/or a wide area network (WAN), and the like.

The indicator 108 may have a speaker (not shown) to alert the patient 106. Further, the indicator 108 may have a microphone (not shown) to allow the patient 106 to input one or more voice commands into the system 116. Further, the indicator 108 may have any suitable input device for inputting data into the indicator 108. For example, the input device may include, but is not limited to, one or more buttons, the microphone, a dial, a touch screen, and the like.

In another embodiment, the indicator 108 may be a clock. The clock may have time settings to alert the patient 106 when it is time to take the medicine 104. The clock may be in communication with the system 116.

The indicator 108 may be linked to a sensor 122 (shown schematically). The sensor 122 may indicate if the medicine container 102 is open and/or closed. The indicator 108 may then send a medicine alert to any of the parties in the system 116 regarding the opened or closed status of the medicine container 102. This opened or closed status may help any of the parties of the system 116 keep track of how many times the medicine 104 is taken, when the last time the medicine 104 was taken, whether a third party (and/or the patient) may be tampering with and/or abusing the medicine 104, and the like.

The patient computer 120 may be any suitable computer, device, phone, pager, and/or personal digital assistant (PDA) that the patient may use to receive an indication and/or alert regarding the medicine. For example, the patient computer 120 may be a cell phone for receiving text messages and/or phone calls regarding a medicine alert. Further, the patient computer 120 may be a smart phone and/or PDA for receiving an email regarding a medicine alert. Although the patient computer 120 is described as a cell phone and/or a PDA, it should be appreciated that the patient computer 120 may be any suitable device for receiving a medicine alert including, but not limited to, a laptop computer, a tablet device, a desktop computer, a pager, and the like. Although, the patient 106 is shown as receiving the medicine alert via the patient computer 120, it should be appreciated that the indicator itself may send the alert directly to the patient 106, for example by an audio and/or visual alert.

In another embodiment, the patient computer 120 may be integral with the indicator 108. For example, the indicator 108 may include the patient computer 120 and may perform any suitable function regarding the system 116 including, but not limited to, those described herein.

The indicator 108 may indicate, and/or alert any of the parties (for example the patient 106, the pharmacy 110, the doctor 112 and/or the third parties 114) in the system 116 about any suitable aspect of the medicine 104. For example, the indicator 108 may be configured to send a medicine alert indicating at what time the patient 106 should take the medicine 104. The medicine alert may indicate the quantity of the medicine to be taken. In addition to sending an indication about when to take the medicine 104, the indicator 108 may send a medicine alert indicating the medicine 104 is running low, when the medicine container 102 has been opened, when the prescription is running out, and the like. The indicator 108 and/or the medicine cover 101 may have an light to allow the patient to see the medicine 104, the indicator 108 and/or the medicine container 102 in the dark.

The system 116 may store, send, distribute, and/or collect data regarding any aspect of the medicine 104, the patient 106, the parties, and/or the medicine alert. For example, the doctor 112 may indicate to the pharmacy 110 a frequency of use, for example how many times a day the medicine 104 should be taken. The pharmacy 110 may then put the frequency on the medicine container 102 and/or send the data to the patient 106, the patient computer 120 and/or the indicator 108. Data regarding the times the medicine is actually taken by the patient may further be stored in the system 116. For example, when the patient acknowledges the medicine alert, the system 116 may record this as a time the medicine 104 was taken. Further, the sensor 122 may alert the system 116 and record the medicine container 102 opening as a time the medicine 104 was taken. Further still, the patient 106 may record the time the medicine 104 was taken. Using the data regarding frequency of use, the system may alert the pharmacy automatically when the prescription is running low. Further, the system may alert the parties in the system when abuse of the medicine 104 may be occurring. Further still, the system 116 may alert the doctor 112 when the prescription for the medicine is about to run out.

There may be a bar code 122 located on the medicine container 101. The bar code 122 as shown is located on the outside of the medicine container 101, although it should be appreciated that the bar code may be located on any suitable surface of the medicine container 101. The bar code 122 may be used by any of the parties described herein to provide information regarding the patient 106, the medicine 104, the times of use, the refill amounts, and the like. The bar code 122 may be used as an identification method for the patient 106 and/or the medicine 104 as will be described in more detail below.

The data stored and used in the system 116 may be stored, collected, manipulated, and analyzed by a medicine data unit 124. The medicine data unit 124 may take the form of an entirely hardware embodiment, an entirely software embodiment (including firmware, resident software, micro-code, etc.), or an embodiment combining software and hardware aspects. The medicine data unit 124 may be located partially and/or wholly anywhere in the system 116 for example, in the indicator 124, the patient's computer 120, at the pharmacy 110, at the doctor 112, within the communication network 118 and the like. Embodiments may take the form of a computer program embodied in any medium having a computer usable program code embodied in the medium. The embodiments may be provided as a computer program product, or software, that may comprise a machine-readable medium having stored thereon instructions, which may be used to program a computer system (or other electronic device(s)) to perform a process. A machine readable medium comprises any mechanism for storing or transmitting information in a form (such as, software, processing application) readable by a machine (such as a computer). The machine-readable medium may comprise, but is not limited to, magnetic storage medium (e.g., floppy diskette); optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions. Embodiments may further be embodied in an electrical, optical, acoustical or other form of propagated signal (e.g., carrier waves, infrared signals, digital signals, etc.), or wireline, wireless, or other communications medium. Further, it should be appreciated that the embodiments may take the form of hand calculations, and/or patient comparisons.

The patient 106 may be any person and/or persons taking the medicine 104. The patient 106 may have trouble remembering when to take the medicine 104 and/or when they have taken the medicine. Further, the patient 106 may want to be discreet about what type of medicine 104 they are taking. The medicine cover 101 may discretely cover the medicine container 102 thereby concealing the medicine and maintaining the patient's 106 privacy.

The medicine 104 may be any suitable medicine that a patient may take. The medicine 104 may be over the counter medicine or prescription medicine. Examples of the types of medicine that may be include, but is not limited to, pain killers, birth control pills, medicine for mental disorders, antibiotics, cholesterol medicine, aspirin, allergy medicine, decongestants, and the like.

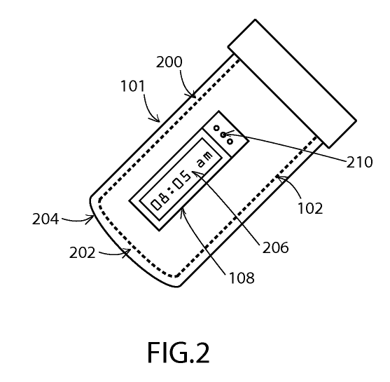

FIG. 2 depicts a perspective view of the medicine cover 101 covering the medicine container 102. As shown, the medicine cover 101 is a cylindrical sleeve 200 connected to a bottom 202. The cylindrical sleeve 200 may be configured to be placed over the bottle portion of a cylindrical medicine bottle, or the medicine container 102. The bottom 202 may be a substantially circular bottom configured to cover the bottom of the medicine container 102. The bottom 202 and the cylindrical sleeve 200 may be one integral piece, or may be connected together using any suitable method including, but not limited to, stitching, gluing, welding and the like.

The cylindrical sleeve 200 may be made of a material, or constructed in a manner, that allows the cylindrical sleeve 200 to radially expand slightly. The radial expansion of the cylindrical sleeve 200 may allow the cylindrical sleeve 200 to grip the medicine container 102 when the medicine container 102 is inserted into the cylindrical sleeve 200. For example, the cylindrical sleeve 200 may have an inner diameter that is slightly smaller than an outer diameter of the medicine container 102 when the cylindrical sleeve 200 is not on the medicine container 102. Therefore when the diameter of the cylindrical sleeve 200 is expanded to cover the medicine container 102, tension (or hoop stress) may be created in the cylindrical sleeve 200 that serves to grip the medicine container 102.

The bottom 202 may be a substantially circular bottom for covering the bottom of the medicine container 102. The bottom 202 may be a solid piece that covers the entire bottom of the medicine container 102. Further, the bottom 202 may be one or more separate pieces configured to prevent the medicine container 102 from falling through the medicine cover 101, but not entirely covering the bottom of the medicine container 102. The bottom 202 may have a flat bottom surface 204. The flat bottom surface 204 may allow the medicine cover 101 to support the medicine container 102 in an upright position. The flat bottom surface 204 may be formed of the same material the bottom and/or cylindrical sleeve 200 is made from, or may be a separate rigid material.

Although the medicine cover 102 is shown with the bottom 202, it should be appreciated that the medicine cover 102 may not have the bottom 202.

The indicator 108 as shown in FIG. 2 is the clock 206. The clock 206 may indicate the current time. Further the clock 206 may send any suitable medicine alerts including those described herein when the patient should take the medicine 104 (as shown in FIG. 1). The clock 206 may have the input device 208 for adjusting the time and/or setting the medicine alerts. The input device 208 as shown is a plurality of buttons 210, although any suitable input device may be used including, but not limited to, those described herein.

The medicine cover 101 may be made of neoprene in an embodiment. The neoprene cylindrical sleeve 200 may allow the medicine cover to be expandable as described above. The entire medicine cover 101 may be made with the neoprene, or portions thereof may be made of the neoprene. For example, the cylindrical sleeve 200 may be neoprene while the bottom 202 may be made from a separate material. Although the medicine cover 101 is described as being made of neoprene, any suitable material or combination of materials may be used to make the medicine cover 101 including, but not limited to, elastic, fabric, cotton, plastic, metal, a polymer, silk, leather, synthetic leather, denim, and the like.

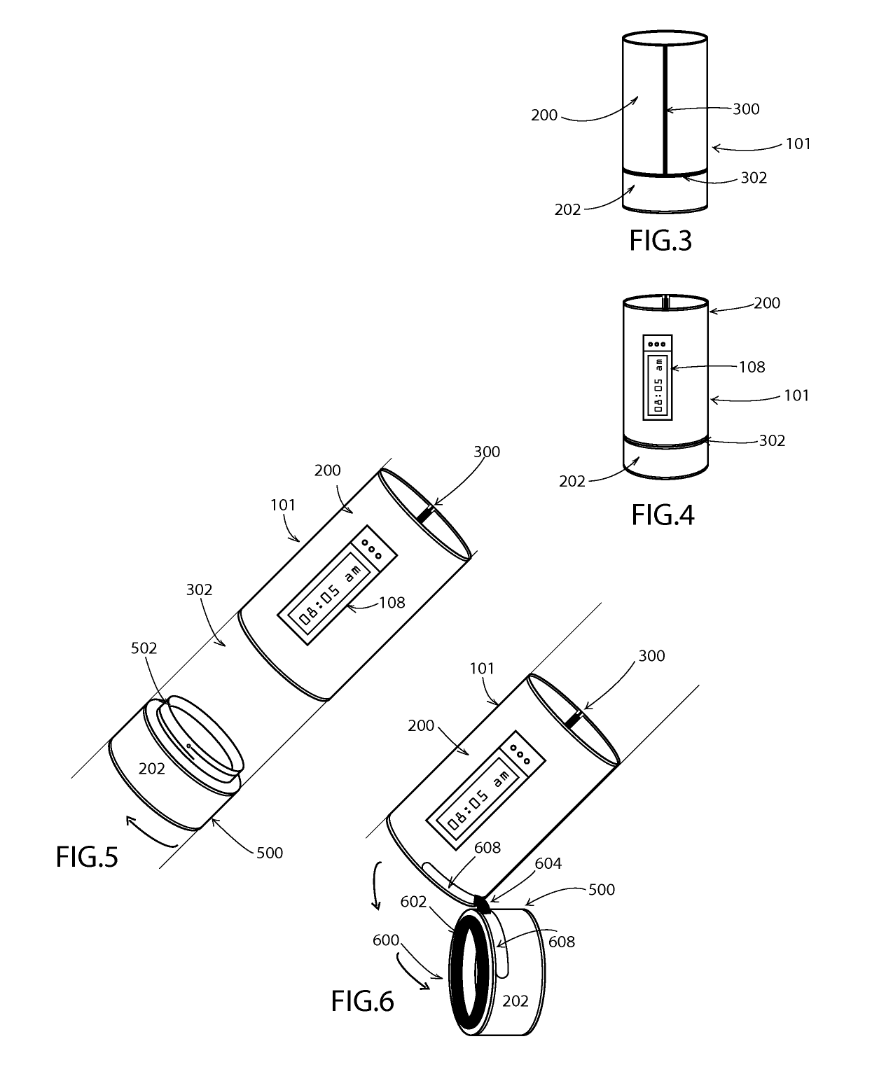

FIG. 3 depicts a back view of the medicine cover 101 without the medicine container 102 (shown in FIGS. 1 and 2). The cylindrical sleeve 200 is shown having a longitudinal seam 300 running the length of the cylindrical sleeve 200. The longitudinal seam 300 may be a permanent connection that holds the cylindrical sleeve 200 in the cylindrical shape as shown. Further, the longitudinal seam 300 may be a fastenable connection that allows the diameter of the cylindrical sleeve 200 to be adjusted. For example, the longitudinal seam 300 may have a Velcro® fastener that allows the cylindrical sleeve 200 to be uncoupled and coupled to fit the size and/or shape of the medicine bottle 102. Further, any suitable fastener may be used including, but not limited to, magnets, buttons, and the like. Further, still it should be appreciated that the medicine cover 101 may not have the longitudinal seam 300 and may be an integral cylinder.

The medicine cover 101 is shown having a bottom seam 302. The bottom seam 302 may form the connection between the cylindrical sleeve 200 and the bottom 202. The bottom seam 302 may be a permanent connection that holds the cylindrical sleeve 200 to the bottom 202. Further, the bottom seam 302 may be a fastenable connection that allows the bottom 202 to be removed from cylindrical sleeve 200. The fastenable connection may be any suitable connection including, but not limited to, a threaded connection, a magnetic connection, any of the connection described herein, and the like.

FIG. 4 depicts a front view of the medicine cover 101 without the medicine container 102 (shown in FIGS. 1 and 2). As shown, the medicine cover 101 has the clock 2-6 as the indicator 108.

FIG. 5 depicts a perspective exploded view of the medicine cover 101 having a removable bottom 500 with a threaded connection 502. The bottom 202 as shown is the removable bottom 500 that may be separated from the cylindrical sleeve 200. The bottom 202 as shown has a male thread configured to secure the bottom 200 to a corresponding female thread on the inside of the cylindrical sleeve 200. Therefore, the patient 106 may remove the bottom 202 as needed by unscrewing the bottom 202 from the cylindrical sleeve 200.

FIG. 6 depicts a perspective exploded view of the medicine cover 101 having the removable bottom 500 with a magnetic connection 600. The bottom 202 as shown is the removable bottom 500 that may be separated from the cylindrical sleeve 200. The bottom 202 as shown has a magnet 602 formed in a circle around the bottom 202. The magnet 602 may be sized to be proximate the bottom end of the cylindrical sleeve 200. There may be another magnet and/or a metallic surface on the bottom end of the cylindrical sleeve 200 to enable the magnet 602 to couple to the cylindrical sleeve 200 thereby engaging the cylindrical sleeve 200 with the bottom 202. Therefore, the patient 106 may remove the bottom 202 from the cylindrical sleeve 200 as needed by applying a force large enough to overcome a magnet force on the bottom 202.

The removable bottom 500 may have a tether 604. The tether 604 may be configured to secure the bottom 202 to the cylindrical sleeve 200 when the bottom 202 is removed from the cylindrical sleeve 200. The tether 604 as shown in FIG. 6 is a flexible cord fastened into a tether groove 606 on outer surface of the bottom 202 and the cylindrical sleeve 200. Although described as a cord, the tether 604 may be any suitable device for securing the bottom 202 to the cylindrical sleeve 200 including, but not limited to, a string, an elastic member, a bungee cord, a wire, a ribbon and the like.

In an embodiment, the medicine cover is sized to fit over a standard sized medicine bottle 102 (as shown in FIGS. 1-3). For example, the medicine cover 101 may have an outer diameter of about 1.5″. Further the longitudinal length of the cylindrical sleeve may be about 2.0″ to 2.25″ in length. It should be appreciated that the dimensions of the cylindrical sleeve 200 and the bottom 202 may be any suitable size so long as the medicine cover 101 substantially covers the medicine container 102. Further, the medicine cover 101 may have varying shapes to accommodate any suitable type of medicine container.

In an alternative embodiment, the medicine cover 101 has a top (not shown) in addition to the bottom 200. The top may be similar to the removable bottoms 500 shown in FIGS. 5 and 6. The top in addition to the bottom 202 may ensure that the entire medicine container 102 is covered by the medicine cover 101.

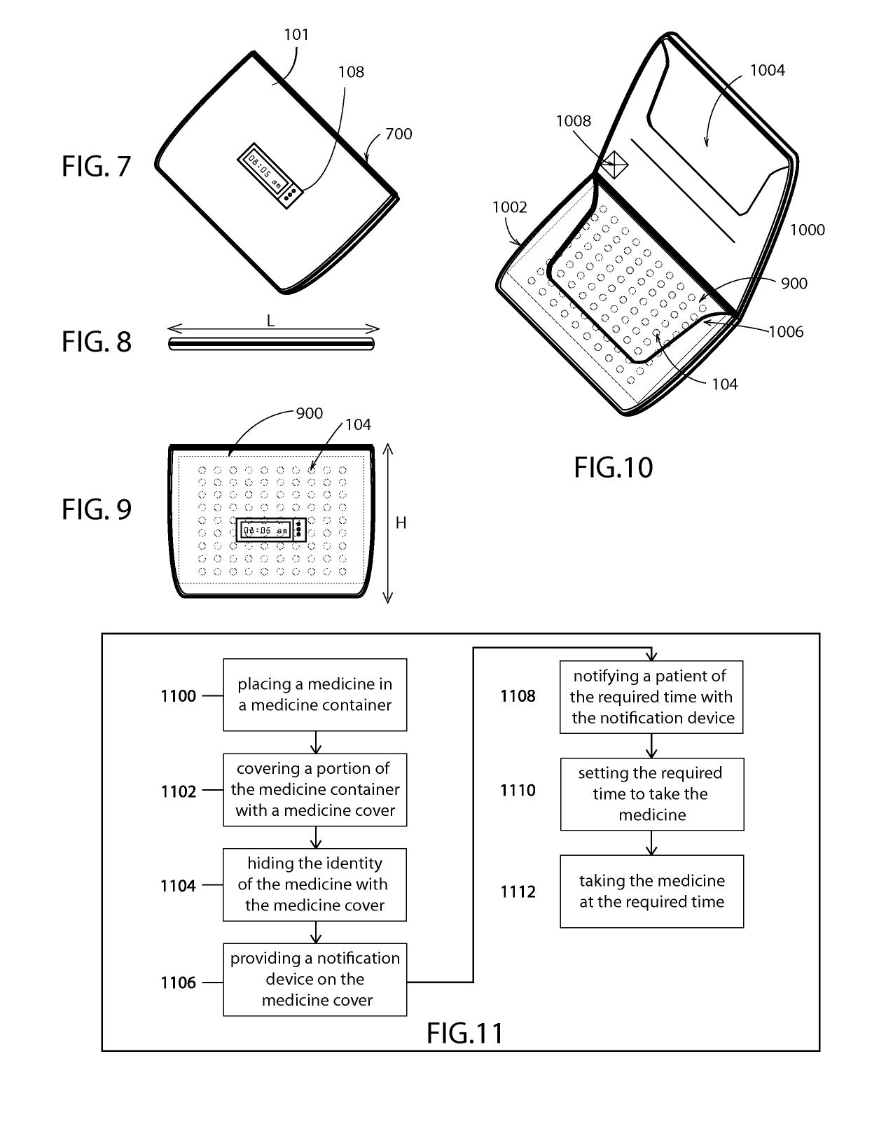

FIG. 7 depicts the medicine cover 101 as a flat medicine cover 700. The flat medicine cover 700 may be used to cover flat medicine, or medicine on a sheet. The flat medicine cover 700 may have a hinged, or foldable, edge configured to allow the flat medicine cover 700 to fold over and enclose the medicine 102. The flat medicine cover 700 may have any of the suitable features and/or system described herein including, but not limited to, the indicator 108, the input device, the system 116, the sensor 122, any suitable device described herein, and the like.

FIG. 8 depicts an end view of the flat medicine cover 700. The flat medicine cover 700 may have a length L. The length L may be any suitable length for enclosing the medicine 104 within the flat medicine cover 700. In an embodiment, the length L may be about 4.375″ although it should be appreciated that the length may vary to any suitable size from 0.25″ to 20.0″.

FIG. 9 depicts a front view of the flat medicine cover 700. The flat medicine cover 700 may have a width W. The width W may be any suitable length for enclosing the medicine 104 within the flat medicine cover 700. In an embodiment, the width W may be about 3.0″ to 3.25″ although it should be appreciated that the width W may vary to any suitable size from 0.25″ to 20.0″. The medicine 104 as shown in FIG. 9 is secured to a sheet 900. Although the medicine is shown as being secured on a sheet, any suitable flat medicine and/or medicine dispenser systems may be stored in the flat medicine cover 700 including, but not limited to, birth control pills, over the counter pills on sheets and the like.

FIG. 10 depicts a perspective view of the flat medicine cover 700 in an open position. The flat medicine cover 700 as shown has a lid 1000 and a medicine holder 1002. The hinged, or foldable, edge may allow the lid 1000 to open to a position that allows the medicine 104 on the sheet to be placed into the medicine holder 1002. As shown, the inside of the lid 1000 may have a pocket 1004. The pocket 1004 may allow the patient 106 (as shown in FIG. 1) to store one or more personal items in the flat medicine cover 700 other than the medicine. The personal items may include, but are not limited to, driver's license, identification, money, credit cards, and the like.

The medicine holder 1002 may have a medicine pocket 1006 on the inside of the medicine holder 1002 portion of the flat medicine cover 700. The medicine pocket 1006 may partially expose the medicine 104 on the sheet 900, as shown, or completely cover the medicine 104 and the sheet 900. By exposing a portion of the sheet 900, as shown, the patient 106 may easily grip and remove the medicine 104 in the flat medicine holder 700. The flat medicine holder 700 may also include a sensor 1008 that communicates with the indicator 108 (as shown in FIGS. 1, 2, 4-7, and 9) and/or the system 116. The sensor 1008 may operate in a similar manner as the sensor 122 (as shown in FIG. 1) and described above.

Any of the medicine covers 101 described herein may have customized designs to suit the patient's taste. The customized designs may include, but are not limited to, color patterns, pictures, paintings, text, and the like.

FIG. 11 depicts a flow diagram depicting a method of using the medicine system as shown in FIG. 1. The flow begins at block 1100 wherein a medicine is placed in the medicine container 102. The medicine container 102 may be any of the medicine containers described herein. The flow continues at block 1102 wherein a portion of the medicine container is covered be the medicine cover 101. The medicine cover 101 may be any of the medicine containers described herein. The flow continues at block 1104 wherein the identity of the medicine is hidden by the medicine cover. The flow continues at block 1106 wherein an optional notification device is provided on the medicine cover 101. The notification device may be any of the indicators 108 described herein. The flow continues at block 1108 wherein the patient is notified of a required time to take the medicine with the notification device. The patient may be notified by any suitable method including, but not limited to, an audio alarm, a visual signal, a text message, an email and the like. The flow continues at block 1110 wherein the required time to take the medicine is set. The required time may be set manually on the clock, or by any suitable method including, but not limited to those described herein. For example, the patient, the pharmacy, the doctor and/or the third parties may send data to the indicator on the medicine cover. The flow continues at block 1112 wherein the patient takes the medicine at the required time. The method may optionally comprise sensing when a patient takes the medicine.

In another embodiment, the patient may order to medicine cover on-line via a company website. The patient may enter data regarding the medicine and/or the medicine system into the website. For example, the patient may enter the type of medicine to be taken, the size of the medicine container, the pharmacy information, the doctor's information, the frequency and/or the required time to take the medicine, and the like. Upon entering the website, the patient may create a personalized log in. The log in may be security protected to prevent third parties from obtaining personal medical data about the patient. The patient upon entering the secured website, may fill in their personal data and any data about the medicine system. The patient may further indicate what type of notification they prefer, for example text messaging, email, audio alert, visual alert, vibration alert, and/or a combination thereof. The patient may further pick any design for the medicine cover, and/or create a new design. The medicine cover may then be sent to the patient customized to the that patient's personal needs and/or wants. The medicine cover may have the bar code configured to allow any of the parties in the medicine system 100 and/or the medicine cover provider to quickly access data regarding the patient and/or the medicine.

The medicine cover may be sent to the patient with a serial code number on a card, which introduces the companies entire product line. The serial code number may be numeric, alphabetic and/or a combination thereof. The medicine system may be configured to only send an indication to the patient for a specific time. When the specific time runs out the patient may need to return to the website and renew the subscription, and/or purchase another medicine cover.

While the embodiments are described with reference to various implementations and exploitations, it will be understood that these embodiments are illustrative and that the scope of the inventive subject matter is not limited to them. Many variations, modifications, additions, and improvements are possible. For example, the techniques used herein may be applied in any container that a user may want to conceal, and/or have an indication of when to use the container, for example on canned and/or bottled beverages.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

What is claimed is:

1. A medicine cover for covering a medicine container, comprising:
a cylindrical sleeve for placing over the medicine container and concealing the identity of the medicine container;
a bottom having an attachment device for securing the bottom to the cylindrical sleeve; and
a medicine for a patient to take that is stored in the medicine container.

2. The medicine cover of claim 1, further comprising a notification device configured to provide a medicine alert to the patient.

3. The medicine cover of claim 2, wherein the medicine alert is when the patient should take the medicine.

4. The medicine cover of claim 2, wherein the notification device is a clock and the medicine alert is an audio alarm heard by the patient.

5. The medicine cover of claim 2, wherein the notification device is in communication with a communication network.

6. The medicine cover of claim 5, wherein the notification device sends the medicine alert to the patient's phone.

7. The medicine cover of claim 5, wherein the notification device sends the medicine alert to pharmacy.

8. The medicine cover of claim 1, wherein the cylindrical sleeve is made of neoprene.

9. An apparatus for concealing a medicine, comprising:
a medicine cover;
a medicine container for storing the medicine; and
an indicator for sending one or more medicine alerts about the medicine.

10. The apparatus of claim 9, wherein the medicine cover further comprises a flat medicine cover having a lid foldable over a medicine holder portion of the flat medicine cover.

11. The apparatus of claim 10, wherein the medicine is on a sheet.

12. The apparatus of claim 9, wherein the indicator is in communication with a medicine system and wherein the medicine system has a communication network and a patient's computer.

13. The apparatus of claim 12, wherein the patient's computer is a personal digital assistant.

14. The apparatus of claim 9, further comprising a sensor configured to indicate when the medicine is being taken.

15. A method for taking medicine, comprising:
placing a medicine in a medicine container;
covering a portion of the medicine container with a medicine cover;
hiding the identity of the medicine with the medicine cover; and
taking the medicine at a required time.

16. The method of claim 15, further comprising providing a notification device on the medicine cover and notifying a patient of the required time with the notification device.

17. The method of claim 16, wherein notifying the patient further comprises sending a text message to the patient.

18. The method of claim 16, wherein notifying the patient further comprises sending an email to the patient.

19. The method of claim 15, further comprising setting the required time.

20. The method of claim 15, further comprising sensing when a patient takes the medicine.
